(19) United States
(12) Patent Application Publication
(10) Pub. No.: US 2003/0055103 A1
(43) Pub. Date: Mar. 20, 2003

(54) Utilization of achyrocline satureoides ("Marcela") extracts and liposomal preparations of natural and semi-synthetic flavonoids for the prevention and treatment of the consequences of stroke and neurodegenerative diseases

(76) Inventors: Horacio Heinzen, Montevideo (UY); Federico Dajas, Montevideo (UY)

Correspondence Address: NEEDLE & ROSENBERG, P.C., The Candler Building, Suite 1200, 127 Peachtree Street, N.E., Atlanta, GA, 30303-1811, US

(21) Appl. No.: 10/190,440

(22) Filed: Jul. 3, 2002

(30) Foreign Application Priority Data
Jul. 4, 2001 (UY) 26.816

(51) Int. Cl. A61K 31/353; A61K 9/127

(52) U.S. Cl. 514/456; 424/450

(57) ABSTRACT

Discovery of a neuroprotective effect in vivo of Achyrocline satureoides (“Marcela”) extracts and of liposomal preparations of natural and semi-synthetic flavonoids structurally related to quercetin. This effect is obtained mainly through antiapoptotic mechanisms, complementary and different of the antioxidant actions of flavonoids. The compounds will be beneficial for the prevention and treatment of stroke and neurodegenerative and aging brain lesions. These benefits will be obtained by the administration of compositions comprising one or various compounds of general formula 1. The liposomal preparation of these compounds increases neuroprotection and will be the preferred application.

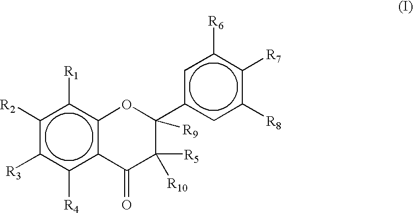

### CROSS-REFERENCE TO RELATED APPLICATIONS

This application claims priority to Uruguayan Patent Application No. 26.816, filed on Jul. 4, 2001, which is herein incorporated by this reference in its entirety.

### FIELD OF THE INVENTION

The invention relates to methods of treating and/or preventing vascular or neurodegenerative brain diseases.

### BACKGROUND OF THE INVENTION

The Importance of Brain Pathology

Vascular and neurodegenerative brain diseases are the most frequent causes of death and morbidity of neurologic origin. With 8% of total deaths and a general incidence of around 2/1000, cerebral pathology is very important because of its high morbidity, the deep affectation of quality of life they provoke and the burden of high socio-economic costs (Reitsma et al. 1998; Kolominsky-Rabas et al. 1998; Samsa et al. 1999; Leppälä et al. 1999). In spite of this situation, there is no specific treatment for neuronal death.

Beyond infectious processes and tumors, brain pathologies that can lead to neuronal death can be globally classified as degenerative and vascular. In the latter, episodes of ischemic and/or hemorrhagic stroke appear over a chronic process of atherosclerosis (Leppala et al. 1999).

Neuronal Death Process in Ischemia

Neuronal death in stroke occurs by complex mechanisms including, among others, metabolic processes, excitotoxicity and oxidative stress (Alexi et al. 2000).

During ischemic stroke there is a decrease of metabolic energy in the form of ATP, affecting membrane ionic pumps (basically Na/K+ATPase) leading to an increase of intracellular Ca++ and Na+ concentrations and to an augmented glutamate release (Nicholls and Attwell, 1990). These factors, mainly the massive Ca++ entry activate catabolic enzymes like proteases, phospholypases and nucleases (Choi, 1995) which can break DNA chains. Proteases that can destroy the cytoskeleton is also activated (Welch et al. 1997). Phospholipase A2, favouring the metabolism of arachidonic acid through lipo-oxygenases, induces the formation of superoxide ion and eicosanoids which in turn increase lipid peroxidation and cellular death. Proteins can be oxidized by free radicals, loosing their primary structure while DNA molecules can suffer mutations leading to carcinogenesis (Halliwell y Gutteridge, 1990, Halliwell, 1990).

Oxidative stress, which is very important in experimental conditions of ischemia-reperfusion, is only part off the complex cellular processes triggered by ischemic ischemia.

Natural Antioxidants and Medicinal Plants for Cerebral Pathology

Since self-maintained oxidative reactions leading to endothelial cell lesions have been identified in atherosclerosis, oxidative stress has been postulated as one of the main causes of clinical symptoms related to this pathology, like myocardial infarction and stroke (Zalba et al. 2001; Kharbanda et al. 2001; Landemmer et al. 2001; Landmesse and Harrison, 2001). For this to occur, the important enzymatic and non enzymatic antioxidant defenses of the organism should be surpassed (Halliwell 1990).

Nevertheless, provision of external antioxidants to regain oxidative equilibrium and to control pathology evolution has failed. Clinical trials with a classical antioxidant like vitamin E did not show beneficial effects on the treatment of cardiovascular pathology risk (Emmett, 1999) and doses higher than 500 mf of ascorbic acid—another natural antioxidant—as well as carotene seem to be detrimental (Colquhoun, 2001; Duthie y Crozier, 2000)

In seeming contradiction with these results, several studies show that natural antioxidants present in fruit, beverages like red wine and beer and plants in general, increase the total antioxidant capacity of blood plasma immediately after their intake (Cao y cols., 1998; Nagyiová y cols., 1998; Riso y cols., 1999; Sung y cols. 2000).

Though there are no controlled studies with these natural antioxidants, evidences from studies of the Mediterranean diet—which would provide a higher amount of them—point to actions different from antioxidant activities to explain the beneficial effect upon cardiovascular risk. The nature of these mechanisms are still unknown (Youdim and Joseph, 2000).

Studies of the relationship between phytochemicals and diet supplements with cerebral protection have been concentrated in compounds originated in Orient, like Gingko biloba or formulations of kampo (Perry et al. 1999; Ondrias et al., 1992).

The rich flora of South America is an important source of medicinal plants. Achyrocline satureoides, a bush popularly known as “marcela” is widely used in folkloric medicine in Uruguay, Argentine and Brazil. The indications for marcela are mainly as digestive and hepatic protector. Some antiviral and anti-inflammatory effects have also been attributed to marcela. (Carney et al., 2002; Ruffa et al., 2002; Kadarian et al., 2002; Zanon et al., 2002; Lorenzo et al., 2000; Garcia, 1999; Hnatyszyn et al. 1999; Santos et al., 1999; De Paula et al., 1998; Desmarchelier et al ., 1998; Rojas et al. 1995; Rocha, 1994; Anesini, 1993; Puhlman et al, 1992; Vargas et al, 1991; Vargas et al. 1990; Simoes et al, 1988; Gutkind et al., 1984).

As far as we know, there are no descriptions of any cerebral action of Achyrocline satureoides.

Beneficial effects of medicinal plants are in general attributed to the antioxidant activity of metabolites like the flavonoids and cumarines, lignanes and xantones (Rice-Evans, 2001; Raty and Das, 1988). The flavonoids are structurally derived from benzo-X-pirone showing a benzenic ring (ring A) linked to an heterocycle of six components (ring C) which is linked to a phenyl group (ring B). The flavonoids are present in plants and are an essential component of human diet. The basic structure of flavonoids is the following.

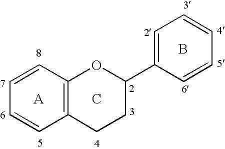

Flavonoid family includes flavones, flavonols, flavanones and flavanols. Substitutions in the basic molecule may imply important changes in the biological activity (Picq et al. 1989; Matter et al. 1992; Chang et al. 1993; Ferriola et al. 1989).

Quercetin

Quercetin is one of the most abundant flavonoids, is widely present in vegetables and fruits, reaching up to 25 mg/person in daily intake in a normal human diet (NTP, 1992). Quercetin is also present in Achyrocline saturcoides, is responsible of some of the brain effects of Gingko biloba (Duthie and Crozier, 2000), and has numerous effects:

antimicrobial (El-Gammal and Mansour, 1986);

antitumoral (Fujiki, et al. 1986);

antithrombotic (Beretz, et al. 1981);

antiviral and antialergic (Vrijsen et al. 1988);

inhibitory of platelet aggregation (Gryglewski et al., 1987);

inhibitory actions against outgrowth of different types of cancer, like ovarian and gastrointestinal cancer (Scambia et al. 1990; Yoshida et al., 1992).

At the cellular level, quercetin inhibits phosphatydil-3-inositol kinase (Agullo et al. 1997; et al., 1992), protein C kinase—PKC—(Ferriola et al. 1989; Picq et al. 1989; Gschwendt et al., 1983), xanthine oxidase (Chang et al., 1993) and NADPH diaphorase (Tamura et al., 1994, Bindoli, et al., 1985).

In spite of these multiplicity of actions, quercetin and flavonoid effects are mainly explained by their antioxidant capacity and their ability to scavenger free radicals. Nevertheless, a clinical study of quercetin did not show beneficial effects (McAnlis et al., 1999) and very little or nothing at all is known about quercetin effects at the level of the central nervous system.

Shutenko et al. (1999) characterized changes in brain nitric oxide levels in a model of global ischemia with reperfusion in the presence of quercetin. Assessing the scavenger activity of quercetin in brain and cerebellar cortex by magnetic resonance these authors have attributed a scavenger action to quercetin. Nevertheless, Shutenko et al. studied the effects of quercetin in an ischemia-reperfusion model, sometimes different from usual clinical situations where there is no reperfusion of the ischemic core, or neurodegenerative diseases that did not occur with significant changes in blood flow.

Another paper has described the beneficial effects of quercetin at an experimental level, on endotoxic shock (Abd El-Gawad and Califa, 2001. The authors explain the effects of quercetin by its antioxidant activity, inhibiting lipoperoxidation and increasing glutathion peroxidase activity. On the other hand, quercetin does not seem to have reversible effects upon metabolic decrease in ischemic conditions (Zager and Ames, 1988).

Beyond antioxidant activity, central nervous system effects of quercetin and flavonoids have been attributed to anti-inflammatory mechanisms and chelation of iron (Juurlink and Paterson, 1998). Recently, Ishikawa et al. (200) have proposed an antiapoptotic activity for quercetin.

In summary, the beneficial effects of medicinal plants are in general referred to their antioxidant activity due to the presence of flavonoids. Effects of quercetin and structurally similar flavonoids upon brain ex vivo are also attributed to their antioxidant action, through inhibition of enzymes like xanthine oxidase or iron chelation.

In vivo neuroprotectant effects of quercetin and flavonoids have not been described yet.

### BRIEF DESCRIPTION OF THE DRAWINGS

FIG. 2. Index of DNA strand breaks assessed by comet assay. (C: control, PC12 cells in culture, no treatment: P, PC12 cells in culture treated with H₂O₂—200 μM, 2 hrs. Exposition). Average Damage index+/−SEM for 6 experiments. Damage index was estimated as the sum of products (damage index: a×number of cells that show a damage, where a goes from 0 to 5) shows a histogram of Index of DNA strand breaks assessed by comet assay (C: control, PC12 cells in culture, no treatment)).

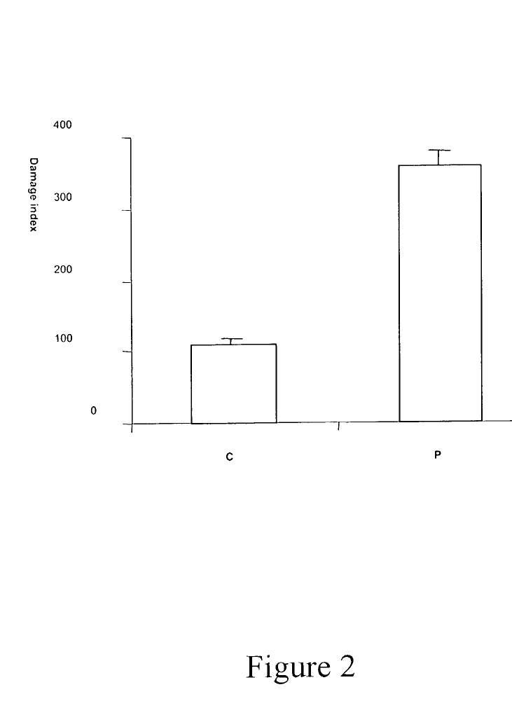

FIG. 3. Caspase inhibitors 3,6 and 9 as well as 3-MT block H₂O₂-induced cell death. (Caspase 6 inhibitor: acetyl-L-valyl-L-glutamyl-L-isoleucyl-L-aspart-1-al, VEID, caspase 9 inhibitor: acetyl-L-leucyl-L-glutamyl-L-histidyl-L-aspart-1-al, LEHD, caspase 3 inhibitor, DEVD). PC12 cells pre-treated with caspase inhibitors (A) and 3-methyladenine, 3-MT, (B) were exposed for 30 min. to 200 μM H₂O₂. Data are expressed as percent of control (mean +/−SEM of three independent experiments ). n=>6 for each point). Highest doses of each compound are significantly different from H₂O₂ treatment and similar to control.—ANOVA and Dunnet—.

FIG. 4. Neuroprotectan effect of quercetin in PC12 cell cultures after H₂O₂ oxidative insult. (%: cell survival expressed as percent of 100% control; C1, DQ are semi-synthetic quercetin derivatives showing no neuroprotectant effect).

FIG. 5. Effects of different flavonoids upon cell survival after treatment of PC12 cells with 200 μM H₂O₂. (cat: catechin; fis: fisetin; K: kaempherol; myr: miricetin; tax: taxifolin; que: quercetin).

FIG. 6. Structures of the flavonoids studied

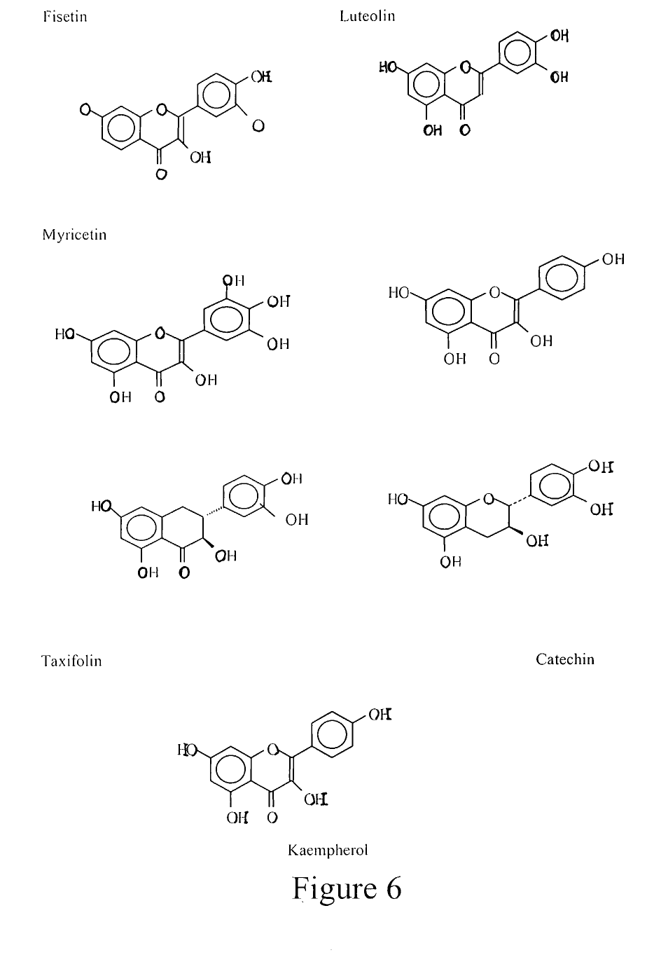

FIG. 7. Shows the Quercetin structure.

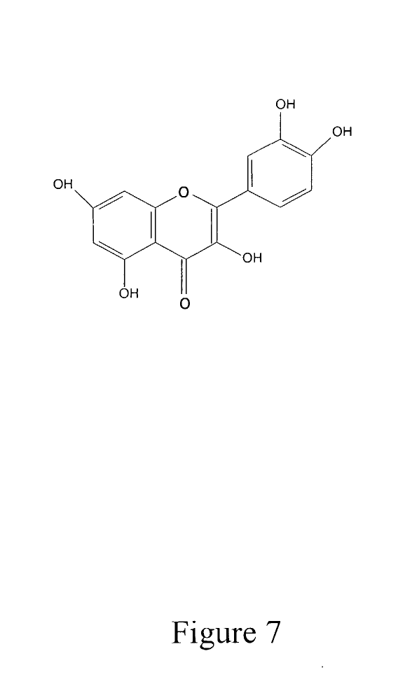

FIG. 8. Shows a bar graph of Effects of aqueous marcela extracts upon cell survival after an H₂O₂ insult (Marcela extracts were added insert rest of caption under Figure). Marcela extracts were added to PC12 cells in culture before they were treated with 200 μM H₂O₂ for 30 min. (P: hydrogen peroxide; C: control; cv: vehicle; pv: hydrogen peroxide and vehicle; ac: different Achyrocline satureoides—marcela—concentrations: 1p=11.5 mg/ml; 2p=8.7 mg/ml; 3p=5.8 mg/ml; 4p=2.9 mg/ml; 5p=1.2 mg/ml; 6p=0.6 mg/ml.—*** p<0.05, Student's t tes t-). As can be observed in FIG. 8, treatment of PC12 cells with different concentrations of aqueous marcela extracts did protect the cells against the H₂O₂ oxidative insult in a dose-dependent manner. Ethanolic extracts did not protect PC12 cells (data not shown).

FIG. 9. Shows a bar graph of Inhibition of spontaneous lipoperoxidation in isolated rat brain membranes by different antioxidants, expressed as IC₅₀ (concentrations required to reach a 50% inhibition).

FIG. 10. Shows a bar graph where Bars represent dopamine level variations expressed as a percent of ipsilateral striatal DA of rats injected with 6-OHDA in the SN and treated systemically with Nicotine, Melatonin, Quercetin, and Boldine; 100% is the concentration of DA of rats injected with 6-OHDA in the SN and treated systemically with the respective vehicle. FIG. 10 shows the results of the same antioxidants of FIG. 9. Nicotine, the weaker antioxidant, reverted dopamine levels in the ipsilateral striatum showing neuroprotectant activity as well as melatonin which is weaker in its neuroprotection. Quercetin and boldine, the strongest antioxidants did not show neuroprotection activity. As a conclusion of these experiments, it can be said that the antioxidant activity of a compound, expressed e.g. by its scavenger potency, is not enough to predict protection of neurons from an oxidative insult.

FIG. 11. Shows a diagram and photos of Upper views of rat brain in ischemia. A: drawing of the brain vasculature pointing to the cerebral medial artery, that is blocked by an intraluminal thread in the experimental conditions. B 24 hs. later, the brain was perfused with tetrazolium salt (TTC) that changed to red in the presence of dehydrogenases, the yellow area shows the ischemic lesion. C: quercetin was administered 30 min. after ischemia. Note the changes in the ischemic area.

FIG. 12. Shows frontal slices of a rat brain submitted to focal ischemia, similar to the one showed in the previous figure, treated with TTC. In B, extended clear-yellow cortico-striatal areas of lesion are observed. Slices showed in A are from a rat treated with intraperitoneal quercetin 30 mins. after the ischemia. The recovery of the ischemic areas is clear cut.

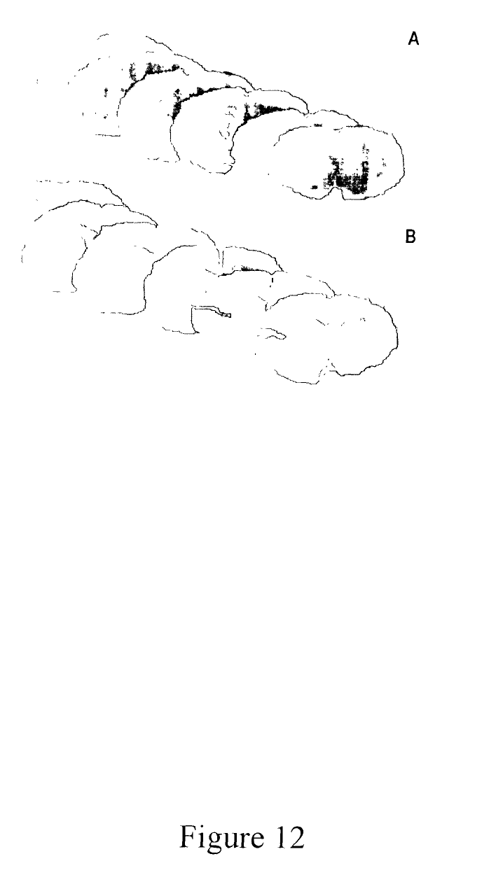

FIG. 13. Shows a graph of Ischemic area assessed in individual frontal slices of rat brain cut in an anterior-posterior axis, as seen in FIG. 2, measured in an imaging computing system (Control:ischemia).

FIG. 14. A bar graph of Volume lesion estimated as per cent of total brain volume assessed using a computer assisted imaging analizer (Control is ischemia in the figure).

FIG. 15. A bar graph of Neuron counts in brain Niss1 stained slices (* p<0.05, paired t test, lesion side compared to contralateral).

FIG. 16. A bar graph of Optical density per gram of wet tissue of brain homogenates after reaction with TTC in ischemic model. Marcela extracts were administered orally 30 min after ischemia and the brains prepared for TTC perfusion 24 hrs. later. (* p<0.05, Student's t test, non paired).

FIG. 17. A graph of Quercetin concentration in rat blood at different periods after intraperitoneal administration.

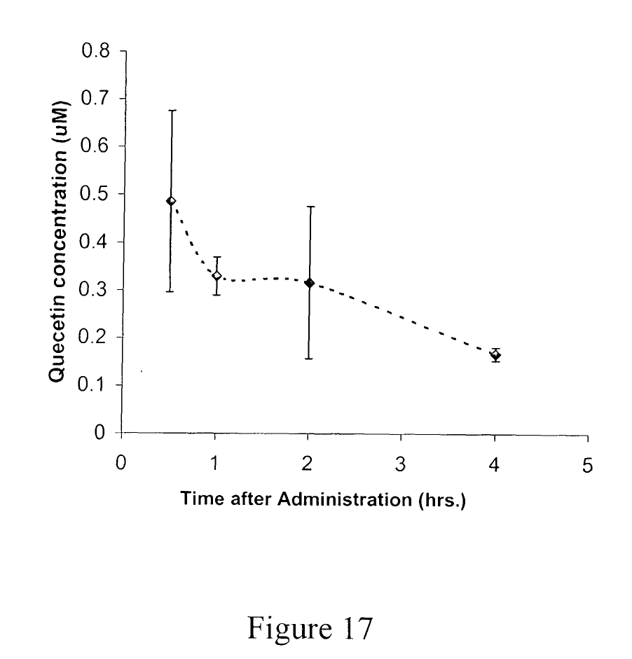

### SUMMARY OF THE INVENTION

The present invention discloses a method of using of flavanoid compounds for the treatment and/or prevention of ischemic-vascular and/or degenerative brain damage comprising administering an effective amount of a composition comprising one or more compounds of formula (I)

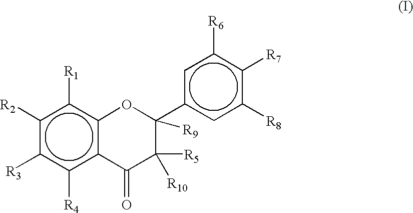

where R1, R2, R3, R4, R5, R6, R7, R8 are indistinctly H, OH, RCOO, RCO, R (R in every case includes alkane, alkene, alkyne or aryl) and R9, R10 are forming a C—C bond are preferred.

### DETAILED DESCRIPTION OF THE INVENTION

In agreement with previous results, we have confirmed that aqueous and ethanolic marcela extracts and flavonoids specially quercetin are potent antioxidants.

The present invention discloses a method of using of flavanoid compounds for the treatment and/or prevention of ischemic-vascular and/or degenerative brain damage comprising administering an effective amount of a composition comprising one or more compounds of formula (I) where R1, R2, R3, R4, R5, R6, R7, R8 are indistinctly H, OH, RCOO, RCO, R (R in every case includes alkane, alkene, alkyne or aryl) and R9, R10 are forming a C—C bond are preferred.

Compounds with the general formula I, in its pure form, or mixtures of them,

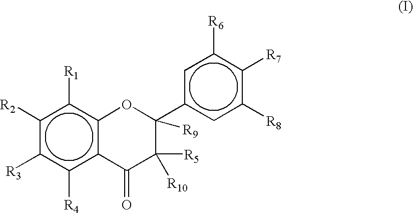

their extracts or some purified or enriched form of them, where R1, R2, R3, R4, R5, R6, R7, R8 are indistinctly H, OH, RCOO, RCO, R (R in every case includes alkane, alkene, alkyne or aryl) and R9, R10 are forming a C—C bond are preferred. The following compounds of the general formula are particularly preferred when R2, R4, R5, R6, R7 are OH. Also R8 can be OH. Alternatively, when R2, R5, R6, R7 are OH or OH, RCOO, RO, (R in every case includes alkane, alkene, alkyne or aryl) and R4 is hydroxy, are specially preferred. The compounds with the general formula I are specially preferred when R2 and R3 are R, RCO, carbohydrate and R2, R4, R5, R6, R7, R8 are indistintinctly H, OH, RCOO, RCO, R (R in every case includes alkane, alkene, alkyne, aryl, or carbohydrate) and R9, R10 are forming a C—C bond. Particularly preferred are the compounds resulting from the above-mentioned combinations, when R6 and R7 are OH. These natural flavonols or semi synthetic modifications of them like quercetin, myrecetin, rutin, rhamnetin, isorhamnetin and their esters, ethers, glycosides, C-acyl, C-alquil, C-alquenyl derivatives are also specially preferred.

The compounds with the general formula where R5═H and R2, R4, R6, R7 are indistintinctly H, OH, RCOO, RCO, R (R in every case includes alkane, alkene, alkyne, aryl, or carbohydrate) and R9, R10 are forming a C—C bond are natural flavones or semi synthetic derivatives of them like luteolin, apigenin, pinocembrin. Particulary preferred are those combinations where R6 and R7 are OH.

When R9 and R10 are H the resulting naturally occurring flavanones and flavanonols or their semi synthetic derivatives with the before mentioned combination of substituents are also preferred.

The pharmaceutical preparations to which this invention refers, are those where one or some compounds of general formula I, (unchanged or their salts) Na+, K+, Ca++, Mg++ and similar) is/are dispersed into a phospholipid, forming liposomes. Natural or semi synthetic modified lecithins are the preferred phospholipids to prepare the liposomes. The pharmaceutical preparations include every one by which this dispersion can be properly given to patients. They include oral, rectal, parenteral, (including subcutaneous, I.M., I.V, intradermic). The preparation of these forms can be done through any techniques or technology employed in pharmacy. These methodologies include any technology needed to obtain a suitable combination of lecithins and flavonoids in defined proportions with the excipients employed in the selected pharmaceutical form. Parenteral preparations, mono or multi doses are specially preferred.

### EXAMPLES

Methods

Chemicals and Reagents

Halothane (Fluothane, Astra-Zeneca), TTC (2,3,5,-triphenylltetrazoliumchloride) and HEPES were obtained from Sigma (St. Louis, Mo., U.S.A.). All others chemicals were of highest commercially available purity and were purchased from Baker (Phillipsburg, Pa., U.S.A.)

Preparation of the Liposomal Quercetin

50 mg quercetin and 123 mg lecithin were dissolved in 20 ml chloroform and the mixture roto evaporated. The quercetin-lecithin preparation was suspended in distilled water and sonicated during 4 hrs. It was kept in 1 ml aliquots at −20C. up to its use.

Animals and Experimental Protocols

Experiments were carried out using male Sprague-Dawley rats (280-350 g). Animals had access to food and water ad libitum, and were housed in groups of six in a temperature controlled environment on a 12 h light/dark cycle. Twenty animals were divided into four groups and injected intraperitoneally (i.p.) 30 min after to MCAo as follows:

Group 1: L/Q was administered in doses of 30 mg/kg (n=6).

Group 2: lecithin (n=5) at the same doses than Group 1

Group 3: An ischemic group was administered with NaCl 0.9% (n=5)

Group 4: a control group injected only with with NaCl 0.9% (n=4).

Permanent Focal MCA Occlusion

Animals were anaesthetized by inhalation of Halothane, in a mixture of oxygen and air through a facemask. During the operation, the body temperature of the animals was continuously monitored at the beginning of the surgical preparation and throughout the experiment with a rectal thermometer., and maintained at 37.5° C. with a heating pad. Permanent focal cerebral ischemia was induced as described by Sydserff et al., 1995^{a} with minor modifications. In brief, a surgical midline incision was made to expose the left common, internal and external carotid arteries. The external carotid and the common carotid arteries were closed by a ligature, the occipital artery was cut by diathermy using a coagulator and the internal carotid artery was temporarily occluded using a micro-aneurysm clip. A small incision was then made in the common carotid artery, and a 19-mm length of 4-0 monofilament nylon suture, its tip rounded by heating, was introduced into the internal carotid artery. The occluding filament was advanced to close the origin of the middle cerebral artery. The anaesthesia was then discontinued, and the animals were returned to their cages after the surgery.

Morphometric Measurement of Infarct Volume

Twenty-four hours after permanent MCAo, the animals were re-anaesthetized with urethane (1.2 g/ml) and intracardially perfused with 100 ml of NaCl 0.9% and 60 ml de 2% TTC (2,3,5,-triphenylltetrazolium chloride) solution. Their brains were quickly removed and them placed in 4% paraformaldehyde and 2% glutaraldehyde for 48 h. Twenty serial coronal sections from each brain were cut in a vibratome (500 μm), beginning from the anterior pole. Digital image were captured from the stained coronal sections using a flatbed colour scanner and analysed using a computer-assisted image analyser (JAVA, Jandel Scientific Software). Total infarct volume was calculated by integrating the area of infarction of all 20 sections (area of infarct in mm 2×thickness of section). Results were expressed as percent of section volume to total volume.

Histology Evaluation

The brains of twelve rats submitted to MCAo were evaluated by histological procedures 24 hrs after occlusion. The animals were anaesthetized with urethane (1.2 g/ml) and intracardially perfused with 100 ml of NaCl 0.9% and 150 ml of 4% paraformaldehyde—2% glutaraldehyde. The brains were quickly removed and placed in 30% Sucrose for at least 48 h. Afterwards, they were frozen in liquid nitrogen and coronal serial sections were cut in a cryostat (6 μm). Sections were stained with hematoxylin and eosin.

Morphological neuronal damage (nuclear hypercromasia with retraction, nuclear fragmentation and marked decrease of cellular population and brain oedema) were chosen as indicators of ischemic tissue damage and were observed separately in striatum and cortex. According to the extension of neuronal damage in each striatum, a semi-quantitative scale was constructed. The striatum divided in quarters, a value of 0 was assigned to the section when no neuronal damage was observed; 1 when neuronal damage occurred in an extension smaller than 1 quarter; 2, neuronal damage occupying up to 3 quarters, 3 damage compromising more than 3 quarters. Division in four segments was taken as equivalent to the four striatal quarters.

To assess the degree of oedema in the lesioned side, the diameter of each hemisphere was measured macroscopically on the slide and the percent difference estimated.

Cell Culture

PC12 cells (Greene and Tischler 1976, Shafer and Atchison 1991) were grown in RPMI supplemented with 10% heat-inactivated horse serum (HSI), 5% fetal bovine serum (FBS), penicillin and streptomycin on a collagen matrix. Typically, cells were sow (1:4 in a total volume of 10 ml complete medium) in 100×20 mm cell culture dishes and grown at 37° C. in at atmosphere of 5% CO₂ for a week. Media was changed twice a week. Cultures were not used in more than 25 passages.

Cells were plated at a low density (100 cells/μl) in 200 μl of complete medium on 96-well cell culture plates on a poli-D-lysine matrix to do all the experiments except the comet assay. To do the latter, 100×20mm-cell culture dishes were adequate. All the experiments were started 24 hrs after the sow.

H₂O₂ Treatment

Cells were exposed to H₂O₂ 200 μM during half an hour (medium was changed to stop the treatment) and all the evaluations were fulfilled 2 hrs later. Recently unfrozen cells present a variable susceptibility to it, so, to make comparisons possible, a susceptibility curve was performed immediately before the experiment and all the experiments (including H₂O₂ controls) were carried at the dose which produced 50% of cell death 2 hrs later.

Cell Viability: MTT Reduction Assay

This assay measures the reduction of 3-(4,5-dimethyl-thiazol-2-yl)-2,5-diphenil tetrazolium bromide (MTT) (yellowish) to formazan (blue) by mitochondrial dehydrogenases of the living cells. The fresh complete medium added after the treatment was supplemented with MTT (0.5 mg/ml ). Following an incubation period of 2 hrs, it was removed and half-its volume of dimethylsulfoxide (DMSO) was added to achieve solubilization. Absorption values were read immediately at 570-640 nm (Denizot and Lang 1986). In spite of the fact that the absolute absorbance values were slightly different, the % of absorbance respect the control remained the same.

Cell Viability: Free LDH Activity

The protocol to measure free lactate dehydrogenase (LDH) released to the medium by cells with broken membrane was modified to use small volumes from Koh and Choi (1987) by mixing 2 hrs after the treatment, 80 μl of supernatant of the treated cells with 120 μl of “LDH buffer” (0.1M phosphate buffer pH 7.5 at 37° C. containing 300 μM NADH and 240 μM pyruvate). NADH absorbs at 340 nm. The slope of NADH consumption was automatically calculated by Revelation software linked to the microplate reader.

The calibration curve was built by sewing 20.000, 15.000, 10.000 and 5.000 cells/well and lysing all of them with triton-X-100 at a final concentration of 0.2%.

DNA Single and Double-Strand Breaks: Comet Assay

Sample Preparation

Microscopic slides were pretreated with 1% NMPA in PBS evenly over the surface of the slide and letting it air-dry. Cells were harvested mechanically and 20 μl of the cell suspension were mixed with 80 μl of 0.75% LMPA in PBS kept at 37° C. Immediately after mixing, 80 μl of the suspension was pipetted on to the pretreated slide, covered with parafilm and left at 4° C. for 5 min. Then the parafilm was removed and the slide was immersed in ice-cold lysis solution (2.5 M NaCl, 100 mM EDTA, 10 mM Tris-HCl and 8 g of NAOH, adjusted to pH 10, to which 1% triton-X-100 and 10% DMSO was added one hour before using) and kept at 4° C. for at least 1 hour (and up to 15 days).

Electrophoresis

The slides were put in the electrophoresis chamber and covered with cold electrophoresis buffer (300 mM NaOH and 1 mM EDTA-final concentrations-, kept separated until one hour before using, when the stocks are mixed and pH is adjusted to 13) for 20 min. The electrophoresis was run at 25 V for 20 min. The volume of buffer was adjusted to have a current from 250 to 300 mA.

Neutralization

The slides were taken from the electrophoresis chamber and washed three times (5 min each) with neutralization buffer (0.4Mtris-HCl pH 7,5). Then they were washed with distilled water.

Staining

The slides were stained with 50 μl of ethidium bromide (20 μg/ml) and covered with a coverslip.

Analysis

The DNA was observed under a fluorescence microscope) and classified according to increasing degree of damage in 6 categories: from 0 to 5. So there was a total of 100 cells counted per treatment per experiment. A DNA-damage index (DDI) was calculated as:

DDI=Σ^{a=5} (number of cells with damage degree a×a).

For references, see Klaude et al (1996) and Tice et al (1996).

Caspases 3, 6 and 9 Inhibition

DEVD, VEID, and LEHD were added 30 min before the treatment with hydrogen peroxide. The dilutions were prepared in 40× concentrations, in DMSO 50%. The doses were diminished in successive experiments until dose-response was observed, except in the case of DEVD because the quantity left was insufficient.

Autophagy Inhibition: 3-MA

In an analogous way, 3-MA (prepared in medium) was added 30 min before the treatment with hydrogen peroxide and the doses were diminished in successive experiments until dose-response was observed.

### EXAMPLE 1

### Decrease of Lipoperoxidation

Aqueous extracts of marcela and quercetin decreased malondialdehyde production during spontaneous lipoperoxidation of rat's brain membranes.

Results are shown in the following Table (Table 1, estimated as IC50). Expressed in quercetin equivalents, marcela IC₅₀ is 40% bigger. (1.56 μg/ml marcela have the same effect than 1.11 μg/ml of quercetin).

TABLE 1

| Compound | IC₅₀ (μM) |
|---|---|
| Quercetin | 3.7 |
| Rutin | 19.2 |
| Catequin | 28.3 |
| Caffeic acid | 48.6 |
| Boldine | 12.75 |
| Melatonine | 1400 |

### EXAMPLE 2

Among various antioxidants assessed (vitamin E, trolox, boldine, etc.) only marcela extracts, quercetin or structurally similar flavonoids protected PC12 cells from a hydrogen peroxide oxidative insult.

Since hydrogen peroxide induces a programmed cell death in our experimental conditions, marcela extracts and quercetin, quercetin derivatives and structurally related flavonoids do protect cells by antiapoptotic mechanisms additional to antioxidant activity. experiments supporting this assumption are:

The addition of hydrogen peroxide (H₂O₂) to PC12 cells in culture in particular conditions where only 50% of the cell population dies, induces a programmed type of cell death. The lack of increase of the enzyme lactic dehydrogenase (LDH) and the increase in DNA strand breaks after H₂O₂ treatment, assessed by the comet assay, would be confirming this assumption.

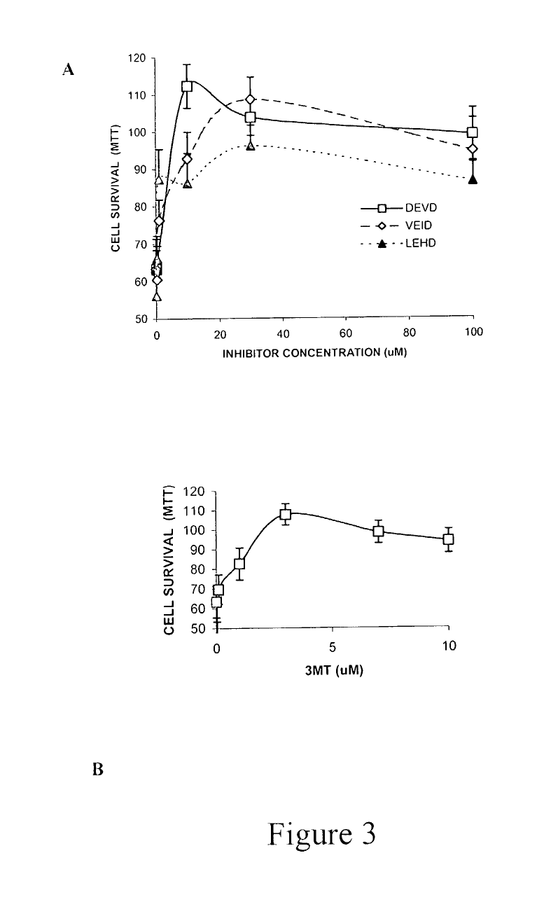

In the same experimental conditions, as shown in FIG. 3, PC12 cell death is inhibited by caspase inhibitors. Caspases are inhibitors of intracellular proteases that once activated trigger programmed cell death (apoptosis). Apoptotic death is therefore the main cause of cell death in the experimental conditions utilized by us.

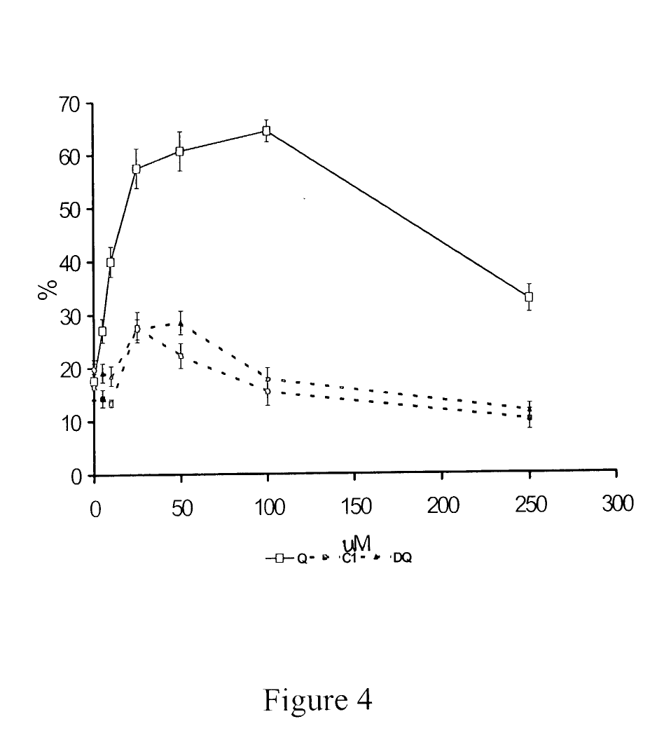

In these conditions—an as shown in FIG. 4—quercetin protects PC12 cells against H₂O₂. Other antioxidants like alpha-tocopherol, boldine, melatonin, etc, did not increased survival in the same conditions. Marcela extracts and flavonoids structurally similar to quercetin are also inhibitors of cell death, inducing the survival of more than 60% of cells. Compounds C1 and DQ,—shown in FIG. 4—were modified in ortocathecholic position of B ring and in the enol of C ring of quercetin, modifications that changed the neuroprotectant effect.

Results obtained with caspases inhibitors allow us to assume that protection given by quercetin against H₂O₂ oxidative damage is mainly due to antiapoptotic mechanisms, beyond antioxidant actions.

The importance of structure on quercetin actions were also assessed. Several flavonoids, structurally related to quercetin: myricetin, kaempherol, taxifolin, catechin, luteolin and fisetin, were tested in their capacity to increase cell viability after H₂O₂ insult.

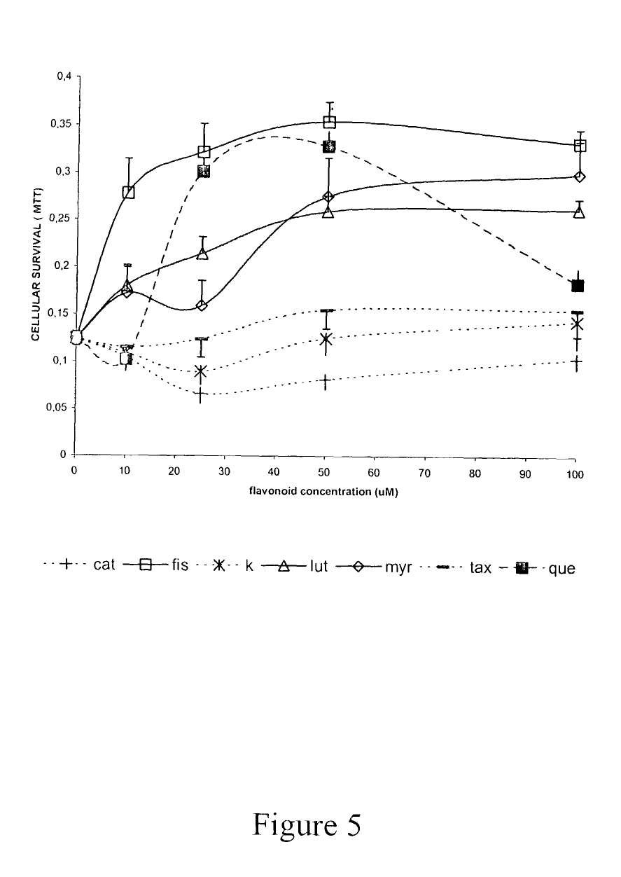

As can be seen in FIG. 5, fisetin, luteolin and myricetin did show neuroprotectant capacity.

These results would indicate that three structural characteristics of flavonoids: catechol of ring B; double bond in position 2,3; oxo function in ring C plus additional hydroxyl groups in positions 3 and 5 are critical for neuroprotectant action. Similar structural characteristics are important for antioxidant activity.

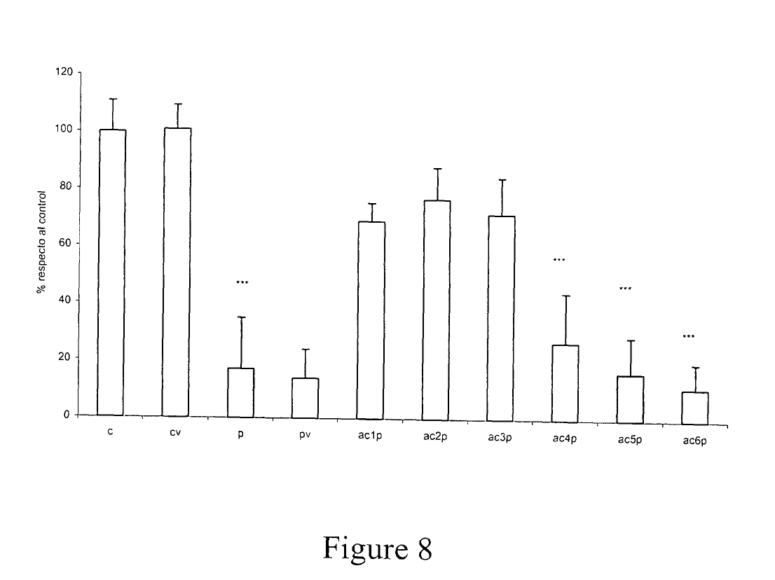

As can be observed in FIG. 8, treatment of PC12 cells with different concentrations of aqueous marcela extracts did protect the cells against the H₂O₂ oxidative insult in a dose-dependent manner. Ethanolic extracts did not protect PC12 cells (data not shown).

### EXAMPLE 3

In spite of its antioxidant potency in vitro and neuroprotectant actions in cell cultures, quercetin—and other antioxidant molecules like boldine—did not revert in vivo the oxidative lesion in an model of parkinsonism.

Accordingly, aqueous solutions of quercetin appear not to have neuroprotectant effects in vivo experimental evidences supporting this assumption are:

Lesion of dopaminergic neurons in the Substantia Nigra (SN) by local microinjections of 6-hydroxydopamine (6-OHDA) is one of the most widely utilized models for experimental Parkinson's Disease. Injection of 6-OHDA induces neuronal death in SN and concomitant loss of terminals in regions innervated by these neurons like the striatum. Since these terminals are dopaminergic, assessment of dopamine in the striatum after 6-OHDA in the SN gives an idea of the extent of the lesion. Boldine, quercetin, melatonin and nicotine were given intraperitoneally before 6-OHDA and the lesion assessed eight days after. The antioxidant capacity of the molecule studied was assessed in the anti-lipoperoxidation assay. The relative potency is shown in the next figure where boldine had the highest antioxidant potency, followed by quercetin and melatonin. Nicotine is not shown because its antioxidant capacity is negligible.

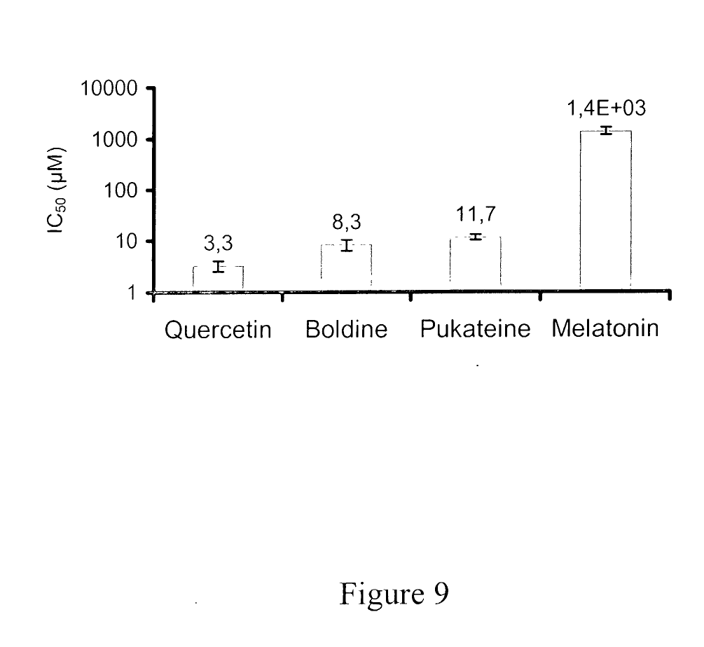

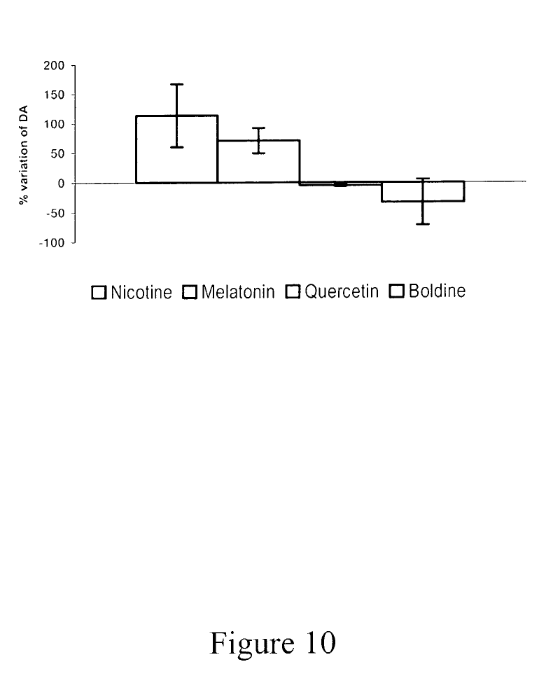

FIG. 10 shows the results of the same antioxidants of FIG. 9. Nicotine, the weaker antioxidant, reverted dopamine levels in the ipsilateral striatum showing neuroprotectant activity as well as melatonin which is weaker in its neuroprotection. Quercetin and boldine, the strongest antioxidants did not show neuroprotection activity.

As a conclusion of these experiments, it can be said that the antioxidant activity of a compound, expressed e.g. by its scavenger potency, is not enough to predict protection of neurons from an oxidative insult.

### EXAMPLE 4

### Quercetin in a Liposomal Preparation, Structurally Related Flavonoids and Marcela Extracts do Protect Brain Neurons in a Model of Focal Permanent Ischemia In The Rat

Neuroprotection by Intraperitoneal Administration of Quercetin

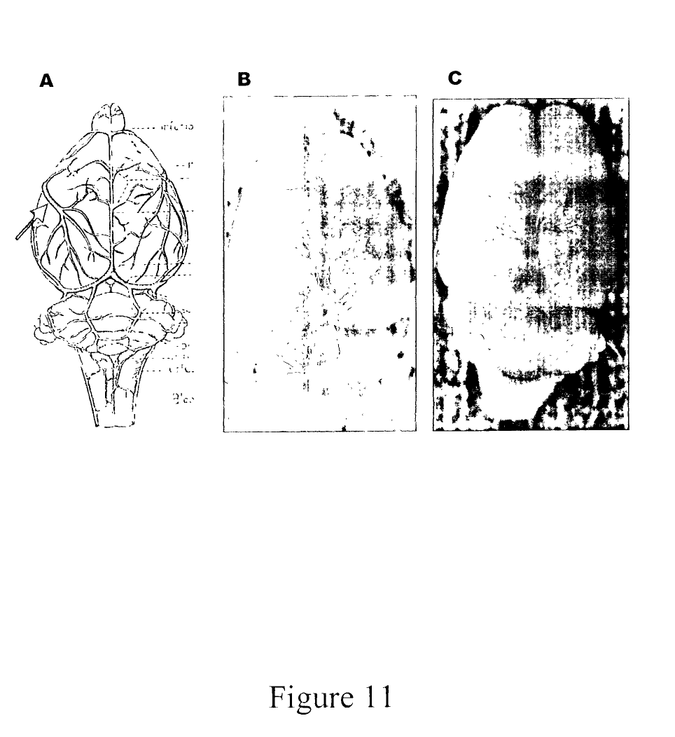

In FIG. 11 (A) a drawing of the rat brain vascular network is shown, the arrow indicating the medial cerebral artery. This artery was occluded in rats by an intraluminal thread and a single dose of 30 mg/kg of a liposomal preparation of quercetin was intraperitoneally administered 30 min after.

In a similar experiment, marcela extracts were given orally in a 300 mg/kg dose. In quercetin experiments, rats were sacrificed 24 hrs. after ischemia and the brains were perfunded with a tetrazolium salt (TCC). TTC changes to red color under the action of mithocondrial dehydrogenases. Accordingly, not colored areas correspond to ischemic regions. (B in FIG. 11). Treatment with quercetin clearly decreased the lesion area (C, FIG. 11).

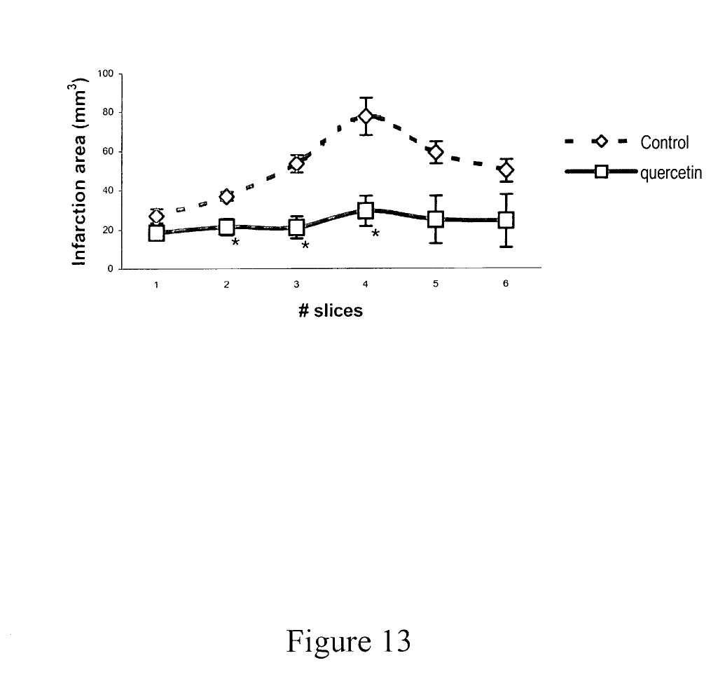

The following FIG. 13 shows ischemic lesion area measured in slices in the antero-posterior axis (control in the figure) and the same measures after quercetin. There was a marked and significant decrease of the ischemic area.

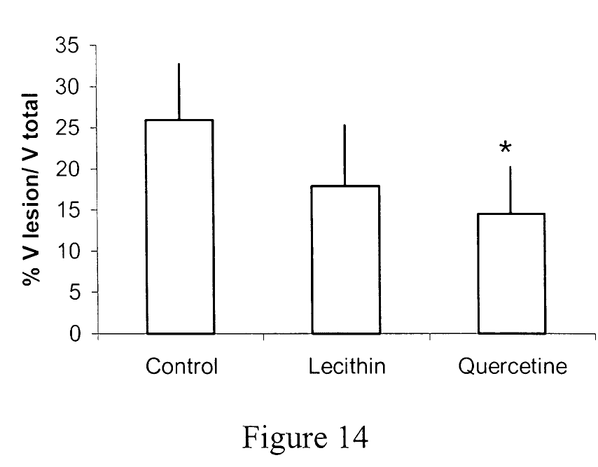

Assessed by computational means, the volume of the ischemic lesion decreased significantly after liposomal quercetin treatment. Lecithin (liposomes) did not change significantly the lesion volume (FIG. 14).

The decrease of the lesion area and volume corresponded to a decreased edema (estimated by brain diameter in brain slices) and to neuronal survival as observed in brain slices stained with Niss1 and Hematoxilin.eosin techniques.

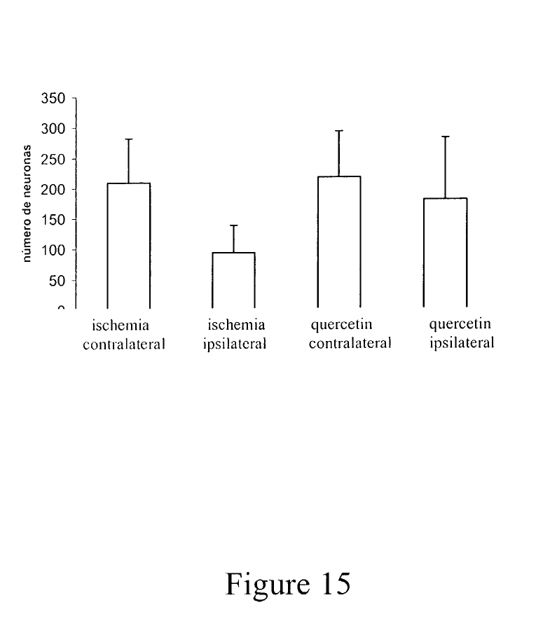

Counts of neurons in Niss1 stained slices showed a significant decrease of neuron number after ischemia in striatum and cortex, compared with the contralateral normal side (FIG. 15). The number of neurons in quercetin-treated rats was higher than ischemic rats and non different from controls.

Neuroprotection by Aqueous Extracts of Marcela

In the case of marcela, the assessment of the protective effects was performed with a minor variant of the technique utilized for quercetin: changes in TTC color were assessed spectrophotometrically instead of microscopically in slices.

Experimental groups without ischemia (n=5) or with ischemia received physiological solution (n=5), aqueous (n=4) or ethanolic (n=3) marcela extracts orally in a 300 mg/kg doses, 30 min after the starting of the ischemic process.

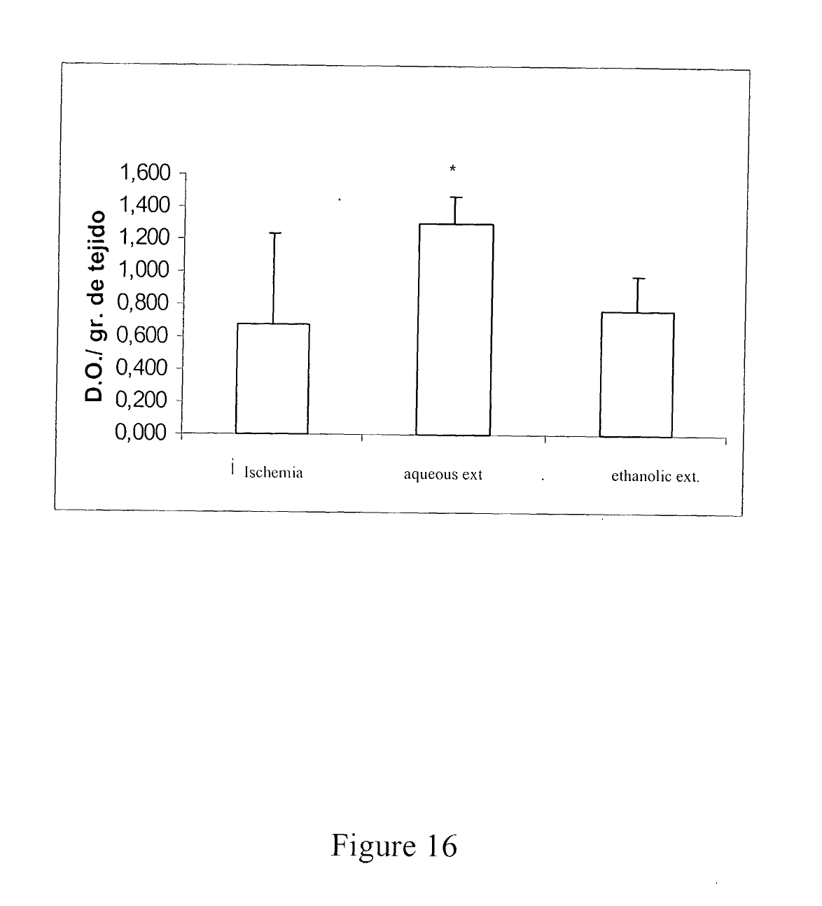

An increase in optical density, reflecting mitochondrial and neuronal survival in ischemic striatum was observed after administration of aqueous extracts of marcela (FIG. 16).

Quercetin concentration of extracts was:

Aqueous: 22 mg quercetin/g extract.

Ethanolic: 16 mg quecetin/g extract.

Quercetin is detected in blood up to two hrs. After intraperitoneal administration

Acute toxicity experiments did not show any adverse effects of quercetin (intraperitoneal or oral) in dosis up to 300 mg/ kg

Animals treated intraperitoneally with aqueous or ethanolic extracts of marcela in doses up to 300 mg/kg showed itching as the only symptom starting with 90 mg (latency 12 min.) and with 180 and 300 mg. No other symptoms were observed.

Rats and mice receiving marcela extracts orally in doses up to 300 mg/ kg, did not show any clinical manifestation of toxicity.

### EXAMPLE 5

### Preparation of an Enriched Extract of Flavonoids from Achyrocline satureoides

Inflorescence of Achyrocline satureoides are macerated for 72 h successively with equal volume of ethanol or extracted with ethanol using a Soxhlet or any other continuous extractor, for 10 hr. The solvent is filtrated and evaporated under reduced pressure to one tenth of the original volume. 10% of this final volume of water is added and the resulting solution is extracted continuously or in batch with petrol ether (b.p. 60-70° C.). The aquo-alcoholic solution is diluted with water to the triple of its volume. and extracted consecutively, in batch or continuously, employing equal volume of ethylacetate and afterwards with equal volume of n-butanol. The organic phases can be combined or not. Dissolved in the ehtyacetate solution are mainly compounds with the general formula I, where R1, R2, R3, R4, R5, R6, R7, R8 are indistincly H, OH, methyl, alkyl, alkenyl, acyl and not carbohydrate residues. They are neither C- nor O-flavonoid glycosides. The composition of the butanol solution is the inverse. The solvent is then evaporated under reduced pressure. The compounds thus obtained are structurally related to quercitin, kampferol, luteolin, apigenin and their derivatives.

### EXAMPLE 6

### Semisynthetic Flavonoids

Acyl Derivatives

Acyl derivatives of flavonoids are prepared by any suitable esterification method of those reported in literature, employing acetic, fatty or aryl acid derivatives residues. The esterification reaction is followed by thin layer chromatography and quenched when the desired amount of acyl residues are incorporated.hte mixture is the fractionated through any suitable method (chromatography, crystallization, etc.)

The compounds thus obtained have the general formula I were R1, R2, R3, R4, R5, R6, R7, R8 can be H, OH or RCO, (R=alkyl, alkenyl, alkinyl, or aryl) according to the starting material employed and the desired amount of esterification.

### EXAMPLE 7

### Ether Derivatives

Ether derivatives of flavonoids are synthesized using the suitable alkyl, alkenyl or alkinyl halide using the Williamson synthesis. The amount of ether formation is controlled by regulating the amount of halide and time of reaction.

The compounds thus obtained have the general formula I were R1, R3, R2, R4, R5, R6, R7, R8 can be either H, OH or R (R=alkyl, alkenyl , alkinyl, or aryl) according to the starting material employed and the desired amount of etherification.

### EXAMPLE 8

### C-Derivatives of Flavonoids

C-derivatives of flavonoids are made employing Friedel-Krafts or Fries protocols, using any suitable combination of Lewis acid and alkyl, alkenyl or aryl halide or acyl halide. The fenolic hydroxyls can be protected or not. The compounds thus obtained have the general formula I were R1, R3, are alkyl, alkenyl , alkinyl, aryl or acyl and R2, R4, R5, R6, R7, R8 can be H, or OH, according to the starting material employed and the R employed. Throughout examples 1-4, R9-R10 are C—C.

### EXAMPLE 9

### Preparation of Liposomes

Any suitable methodology for the preparation of liposomes can be employed, at any flavonoid/liposome ratio which yields an O/W or W/O stable suspension.

### REFERENCES

Agullo G.; Gamet-Payastre L.; Manenti S.; Viala C.; Remesy C.; Chap H.; Payastre B. (1997). Relationship between flavonoid structure and inhibition of phosphatidylinositol 3-kinase: a comparison with tyrosine kinase and protein kinase C inhibition. Biochemical Pharmacol. 53: 1649-1657.

Ahkawa, H.; Ohishi, N. Y Yagi, K. (1979): Assay for lipid peroxides in animal tissues by thiobarbituric acid reaction. Analytical Biochemistry, 95:331-358

Alexi, T.; Borlongan, C.; Faull. C.; Williams, C.; Clark, R.; Gluckman, P.; Hughes, P. (2000): Neuroprotective Strategies for basal ganglia degeneration: Parkinson's and Huntington's diseases. Progress in Neurobiology, 60:5:409-470

Anesini C, Perez C. (1993) Screening of plants used in Argentine folk medicine for antimicrobial activity. J Ethnopharmacol. 39: 119-128.

Beretz, A.; Stierle, A.; Anton, R.; Cazenave, J P (1981): Role of cyclic AMP in the inhibition of human plateled aggregation by quercetin, a flavonoid that potentiates the effect of prostacyclin. Biochem Pharmacol. 31: 3597-3600.

Cao C., Rusel R M, Lischner N., Prior R L (1998). Serum antioxidant capacity is increased by consumption of strawberries, spinach, red wine or vitamin C in elderly women. J. Nutr 28:2383.2390.

Carney J R, Krenisky J M, Williamson R T, Luo J. (2002) Achyrofuran, a new antihyperglycemic dibenzofuran from the South American medicinal plant Achyrocline satureioides. J Nat Prod. 65:203-205.

Ceriatti F S, Rovera M, Sabini L J, Ramos B A. (1999) Search for antiviral activity of certain medicinal plants from Cordoba, Argentina. Rev Latinoam Microbiol. 41:59-62. middle focal cerebral ischaemia: a role for apoptosis?. J. Cereb. Blood Flow Metab. 16: 195-201.

Chang W S.; Lee Y J.; Lu F J.; Chiang H C. (1993). Inhibitory effects of flavonoids on xanthine oxidase. Anticancer Res. 13:2165-2170.

Colqhoun D. (2002). Nutraceuticals: vitamins and other nutrients in coronary heart disease. Curr. Opinion in lipidology 12:639-646.

Dajas-Bailador, F.; Martinez, A.; Costa, G., Abin, A.; Martignoni, E.; Nappi, G.; Dajas, F. (1998): Hydroxyl radical production in the substantia nigra after 6-hydroxydopamine y hypoxia-reoxygenation. Brain Research. 813: 18-25.

Das, D.K. (1 994): Naturally ocurring flavonoids: Structure, chemistry y high-performance liquid chromatography methods for separation y characterization. Methods Enzymol. 234: 410-420.

Denizot F., Lang R. (1986) Rapid colorimetric assay for cell growth and survival. Modifications to the tetrazolium dye procedure giving improved sensitivity and reliability. Journal of immunological methods 89, 271-277.

De Paula I C, Ortega G G, Bassani V L, Petrovick P R. (1998). Development of ointment formulations prepared with Achyrocline satureioides spray-dried extracts. Drug Dev Ind Pharm. 24:235-241.

Desmarchelier C, Coussio J, Ciccia G. (1998) Antioxidant and free radical scavenging effects in extracts of the medicinal herb Achyrocline satureioides (Lam.) DC. (“marcela”). Braz J Med Biol Res. 31:1163-1170.

Dewar, D.; Yam, P. Y McCulloch, J. (1999): Drug development for stroke: importance of protecting cerebral white matter. European Journal of Pharmacology. 375: 41-50

Duthie G.; Crozier A. (2000) Plant derived phenolic antioxidants. Curr. Opinion Clin Nutrition and Metab. 3:477-451.

Emmert, D, Kirchner, (1999). The Role of Vitamin E in the prevention of heart disease. Archives of Family Medicine. 8:537-542.

El-Gammal, A.; Mansour, R M A (1986). Antimicrobial activities of some flavonoid compounds. Zentralbl Mikrobiol 141:561-5

El-Gawad A.; Califa H M. (2001). Quercetin, coenzyme Q10 and L-canavanine as protective agents against lipid peroxidation and nitric oxide generation in endotoxin induced shock in rat brain. Pharmacological Res, 43:257-263.

Fujiki, H.; Horiuchi, T.; Yamashita, K. (1986): Inhibition of tumor promotion by flavonoids. In: Cody V. Middleton E. Harborne J B, Eds. Plant flavonoids in biology and medicine: Biochemical, pharmacological and structure-activity relationships. pp 429-440 New York: Alan R. Liss

Garcia G, Cavallaro L, Broussalis A, Ferraro G, Martino V, Campos R. (1999) Biological and chemical characterization of the fraction with antiherpetic activity from Achyrocline flaccida. Planta Med. 65:343-346.

Gryglewski, R. J.; Korbut, R., Robak, J.; Swies, J. (1987). On the mechanism of antithrombotic action of flavonoids. Biochem. Pharmacol. 36:317-322

Gschwendt M.; Horn F.; Kittstein W.-; Marks F. (1983). Inhibition of the calcium and phospholipid dependent protein kinase activity from mouse brain cytosol by quercetin. Biochem Biophys Res. Commun. 117:444-447.

Gutkind G, Norbedo C, Mollerach M, Ferraro G, Coussio J D, de Torres R. (1984). Antibacterial activity of Achyrocline flaccida. J Ethnopharmacol. 10:319-321.

Halliwell, B. (1990): How to characterize a biological antioxidant. Free Radic. Res. Commun 9:1-32

Halliwell, B. Y Gutteridge, J. M. C. (1999): Oxidative stress y ischaemic or traumatic brain injury. In: Free radicals in Biology y Medicine. Third edition (eds.) Halliwell, B V. Gutteridge, J. M. C. Oxford. University Press pp 733-736.

Hanasaki, Y.; Ogawa, S.; Fukui, S. (1994): The correlation between active oxygen scavenging and antioxidative effects of flavonoids. Free Radic. Biol. Med. 16:6:845-50

Hnatyszyn O, Broussalis A, Herrera G, Muschietti L, Coussio J, Martino V, Ferraro G, Font M, Monge A, Martinez-Irujo J J, Sanroman M, Cuevas M T, Santiago E, Lasarte J J. (1999) Argentine plant extracts active against polymerase and ribonuclease H activities of HIV-1 reverse transcriptase. Phytother Res. 13:206-209.

IARC (1983): Monographs on the evaluation of the carcinogenic risk of chemicals to humans. Quercetin. 31:213-29 LYON, France: International Agency for Research on Cancer.

Juurlink B H.; Paterson P G. (1998). Review of oxidative stress in brain and spinal cord injury: suggestions for pharmacological and nutritional management strategies. J. Spinal Cord. Med. 21: 309-334.

Juurlink B H.; Paterson P G. (1998) Review of oxidative stress in brain and spinal cord injury: suggestions for pharmacological and nutritional management strategies. Jr. of Spinal Cord Medicine 21: 309-334.

Kadarian C, Broussalis A M, Mino J, Lopez P, Gorzalczany S, Ferraro G, Acevedo C. (2002) Hepatoprotective activity of Achyrocline satureioides(Lam) D. C. Pharmacol Res. 45:57-61.

Karton Y.; Jiang J L.; Melman N.; Olah M E.; Stiles G L.; Jakobson K A. (1996). Synthesis and biological activities of flavonoid derivatives as A3 adenosine receptor antagonists. Jr. of Medicinal Chem 39:2293-22301.

Kharbanda R K, Deanfield J. E (2001). Functions of the healthy endothelium. Coronary Artery Disease; 12:485-491

Klaude M., Eriksson S., Nygren J. and Ahnström G. (1996) The comet assay: mechanisms and technical considerations. Mutation Research 363, 89-96.

Koh J. and Choi D. (1987) Quantitative determination of glutamate mediated cortical neuronal injury in cell culture by lactate dehydrogenase efflux assay. Journal of Neuroscience Methods 20, 83-90.

Kolominsky-Rabas; Sarti C; Heuschmann P; Graf C; Siemonsen S; Kundoerfer B; Katalinic A; Lang E; Gassmann K; von Stockert T.(1998) A Prospective Community-Based Study of Stroke in Germany—The Erlangen Stroke Project (ESPro): Incidence and Case Fatality at 1, 3, and 12 months. Stroke 29:2501-2506.

Landemmer U.; Harrison D. G. (2001). Oxidative stres and vascular damage in hypertension. Coronary Artery Disease; 12:455-461.

Landmesse U.; Harrison D. G (2001). Oxidant stress as a marker for cardiovascular events: Ox marks the spot. Circulation.; 104:2638-2640

Leppälä J; Virtamo J; Fogelholm R; Albanes D; Heinonen O. (1999) Different Risk Factors for Different Stroke Subtypes: Association of Blood Pressure, Cholesterol, and Antioxidants. Stroke 30:2535-2540

Lorenzo D, Atti-Serafini L, Santos A C, Frizzo C D, Paroul N, Paz D, Dellacassa E, Moyna P. (2000). Achyrocline satureioides essential oils from southern Brazil and Uruguay. Planta Med. 66:476-7.

Matter W F.; Brown R F.; Vlahos C J. (1992). The inhibition of phosphatidylinositol 3-kinase by quercetin and analogs. Biochem Biophys Res Commun. 186: 624-631

Nagyová A., Kidlacková A., Grancicová E., Magálová T. (1998). LDL oxidizability and antioxidative status of plasma in vegetarians. Ann. Nutr. Metab 42:328-332.

Nicholls, D.; Attwell, D. (1990): The release y uptake of excitatory amino acids. Treds Pharmacol. Sci. 11:462-468

NTP Technical Report on the Toxicology y Carcinogenesis Studies of Quercetin in F344/N Rats. NIH Publication No. 91-3140. U.S. Department of Health y Human Services, Public Health service, Research Triangle Park, N.C., U.S.A.

Potterat, O. (1997): Antioxidants y free radicals scavengers of natural origin. Current Organic Chemistry. 1:415-440

Oken B.; Storzbach D M.; Kaye J A. (1998) The efficacy of Gingko Biloba on cognitive functions in Alzheimer Disease. Archives of Neurology. 55: 1409-1415.

Oyama Y.; Fuchs P A.; Katayama N.; Noda K. (1994) Myricetin and quercetin, the flavonoid constituents of Ginkgo Bilova extract, greatly reduce oxidative metabolism in both resting and Ca (2+)-loaded brain neurons. Brain Res. 635:125-129.

Picq M.; Dubois M.; Munri-Silem Y.; Prugent A F.; Pacheco H. (1989). Flavonoid modulation of protein kinase C activation. Life Sci 44: 1563-1571.

Puhlmann J, Knaus U, Tubaro L, Schaefer W, Wagner H. (1992) Immunologically active metallic ion-containing polysaccharides of Achyrocline satureioides. Phytochemistry. 31:2617-2621.

Reitsma, J B; Limburg, M; Kleijnen, J; Bonsel, G J; Tijssen, J G. (1998) Epidemiology of Stroke in the Netherlands From 1972 to 1994: The end of the decline in troke mortality. Neuroepidemiology. 17:121-131.

Rice Evans C. (2001) Flavonoid antioxidants. Current Medicinal Chem. 8: 797-807.

Riso P., Pindler A., Santangelo A., Porrini M. (1999) Does tomato consumption effectively increase the resistanse of lymphocyte DNA to oxidative damage? Am. J. Clin. Nutr. 69:712-718.

Rocha M J, Fulgencio S F, Rabetti A C, Nicolau M, Poli A, Simoes C M, Ribeiro-do-Valle R M. (1994). Effects of hydroalcoholic extracts of Portulaca pilosa and Achyrocline satureioides on urinary sodium and potassium excretion. J Ethnopharmacol. 22:179-83.

Rodriguez J.; Yanez J., Vicente V., Alcaraz M.; Benavente Garcia O.; Castillo J.; Lorente J.; Lozano J A. (2002). Effects of several flavonoids in the growth B16F10 and SK-MEL-1 melanoma cell lines: relationship between structure and activity. Melanoma Res. 12:99-107.

Rojas de Arias A, Ferro E, Inchausti A, Ascurra M, Acosta N, Rodriguez E, Fournet A. (1995) Mutagenicity, insecticidal and trypanocidal activity of some Paraguayan Asteraceae. J Ethnopharmacol. 45:35-41.

Romay, C.; Armesto, J.; Remiréz, D.; González, R.; Ledón, N. Y Garcia, I. (1998): Antioxidant y anti-inflammatory properties of c-phycocyanin from blue-green algae. Inflamm. Res. 47 (1): 36-41

Ruffa M J, Ferraro G, Wagner M L, Calcagno M L, Campos R H, Cavallaro L. (2002) Cytotoxic effect of Argentine medicinal plant extracts on human hepatocellular carcinoma cell line. J Ethnopharmacol. 79:335-339.

Salah, N.; Miller, N. J.; Paganga, G.; Tijburg, L. (1995): Polyphenolic flavanols as scavengers of aqueous phase radicals y as chain-breaking antioxidants. Archives of Biochemistry Biophysics, 232: 2: 339-346

Samsa G; Bian J; Lipscomb J; Matchar D (1999): Epidemiology of Recurrent Cerebral Infarction: A Medicare Claims-Based Comparison of First and Recurrent Strokes on 2-Year Survival and Cost. Stroke, 30: 338-349

Santos A L, Ripoll D, Nardi N, Bassani V L. (1999). Immunomodulatory effect of Achyrocline satureioides (LAM.) D.C. aqueous extracts. Phytother Res. Feb; 13:65-6.

Scambia, G.; Ranelletti, F. O.; Benederri Pacini, P. (1990): Inhibitory effect of quercetin on OVCA 433 cells y presence of type II oestrogen binding sites in primary ovarian tumors y cultured cells. Br. J. Cancer 62: 942-947

Shutenko, Z.; Henry, Y.; Pinard, E.; Seylaz, J.; Potier, P.; Berthet, F.; Girard, P.; Sercombe, R. (1999): Influence of the antioxidant Quercetin in vivo on the level of nitric oxide determined by electron paramagnetic resonance in rat brain during global ischemia y reperfusion. BiochemPharmacol 57:199-208

Sichel, G.; Corsaro, C.; Scalia, M.; Belio, A. J. D.; Bonono, R. P. (1991): In vitro scavenger activity of some flavonoids y melanius against O₂⁻. Free Radic. Biol. Med. 11:1-8

Simoes C M, Schenkel E P, Bauer L, Langeloh A. (1988). Pharmacological investigations on Achyrocline satureioides (LAM.) DC., Compositae. J Ethnopharmacol. 22:281-93.

Sung H., Nah J., Chun S., Park H., Yang S E., Min W K.(2000). In vivo antioxidants effect of green tea. Eur. J. Clin Nutr 54:527.29.

Swanson, R. A.; Morton, M. T.; Tsao-Wu, G.; Saavalos, R. A.; Davidson, C. Y Sharp, F. R. (1990): A semiautomated method for measuring brain infarct volume. J. Cereb. Blood Flow Metab. 10:290-293

Sydserff, S. G.; Cross, A. J. y Green, A. R. (1995^{a}): The neuroprotective effect of chlormethiazole on ischaemic neuronal damage following permanent middle cerebral artery ischaemia in the rat. Neurodegeneration, 4, 323-328.

Tamura M.; Kagawa S.; Tsuruo Y.; Ishimura K.; Morita K. (1994) Effects of flavonoid compounds on the activity of NADPH diaphorase prepared from the mouse brain. Jpn. J. Pharmacol . 665:371-373.

Tice R., Andrews P. Singh N. (1990) The single cell gel assay: a sensitive technique for evaluating intercellular differences in DNA damage and repair. In DNA damage and repair in human tissues (Edited by Sutherland B. and Woodhead A.), p. 291. Plenum Press, New York.

Touzani, O.; Galbraith, S.; Siegl, P. Y McCulloch, J. (1997): Endothelin-B receptors in cerebral resistance arterioles y their functional significance after local cerebral ischemia in cats. J. Cereb. Blood Flow Metab. 17.1157-1165

Vargas V M, Guidobono R R, Henriques J A. (1991) Genotoxicity of plant extracts. Mem Inst Oswaldo Cruz. 86 Suppl 2:67-70.

Vargas V M, Motta V E, Leitao A C, Henriques J A. (1990) Mutagenic and genotoxic effects of aqueous extracts of Achyrocline satureoides in prokaryotic organisms. Mutat Res. 240:13-8.

Vrijsen, R.; Everaert, L.; Boeyé, A.; (1988): Antiviral activity of flavones y potentiation by ascorbate. J. Gen. Virol. 69: 1749-1751

Welch, K. M. A.; Caplan, L. R.; Reis, D. J.; Siesjo, B. K. y Weir, B. (1997): Eds., Primer on Cerebrovascular Diseases, San Diego: Acaddmic Press.xanthine dehydrogenase activity. Pharmacol. Res. Comms. 17: 831-839

Yoshida, M.; Sakai, T.; Hosokawa, N. (1992): The effect of quercetin on cell cycle progression y growth of human gastric cancer cells. FEBS Lett 260: 10-13

Youdim K.; Joseph J. (2001). A possible emerging role of phytochemicals in omproving age-related neurological dysfunctions: a multiplicity of effects. Free Rad. Biol. Med. 30:583-594.

Zager E L.; Ames A.(1988). Reduction of cellular energy requirements. Screening for agents that may protect aganst CNS ischemia. J. Neurosurg. 69:568.579.

Zalba G; San José G.; Moreno M. U.; Fortuño M. A.; Fortuño A.; Beaumont F. J.; Diez. (2001). Oxidative stress in arterial hypertension.m Role of NAD(P)H Oxidase. Hypertension.; 38: 1395-1399.

Zea Longa, E.; Weinstein, P. R.; Carlson, S. y Cummins, R. (1989): Reversible middle cerebral artery occlusion without craniectomy in rats. Stroke 20: 84-91

Zerriola P C., Cody V.; Middleton E. (1989). Protein kinase C inhibition by plant flavonoids. Kinetic mechanisms and structure-activity relationships. Biochem Pharmacol 38:1617-1624.

What is claimed is:

1. A method of using of flavanoid compounds for the treatment and/or prevention of ischemic-vascular and/or degenerative brain damage comprising:
administering an effective amount of a composition comprising one or more compounds of formula I. 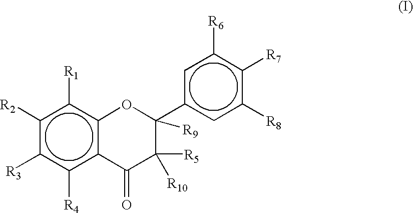
where R1, R2, R3, R4, R5, R6, R7, R8 are indistinctly H, OH, RCOO, RCO, R (R in every case includes alkane, alkene, alkyne or aryl) and R9, R10 are forming a C—C bond.

2. The method of claim 1 wherein the compound of formula I is in liposomal preparations.

3. The method of claim 1 wherein the compound of formula I is naturally-derived.

4. The method of claim 1 wherein the compound of formula I is synthetic.

5. The method of claim 1 wherein the degenerative brain damage is caused by Parkinson's or Alzheimer's disease.
